(19) United States
(12) Patent Application Publication
(10) Pub. No.: US 2017/0020911 A1
(43) Pub. Date: Jan. 26, 2017

(54) ANTITHROMBIN-HEPARIN COMPOSITIONS AND METHODS

(71) Applicant: ATTWILL Medical Solutions, Inc., Hamilton (CA)

(72) Inventors: Leslie Roy Berry, Burlington (CA); Attilio DiFiore, West Jordan, UT (US); Anthony Kam Chuen Chan, Ancaster (CA)

(73) Assignee: ATTWILL Medical Solutions Inc., Hamilton (CA)

(21) Appl. No.: 15/130,441

(22) Filed: Apr. 15, 2016

Related U.S. Application Data
(60) Provisional application No. 62/148,112, filed on Apr. 15, 2015.

(51) Int. Cl. A61K 31/727; A61K 47/48

(52) U.S. Cl. CPC A61K 31/727; A61K 47/48246

(57) ABSTRACT

Compositions and methods for preventing thrombogenesis can include antithrombin and heparin. In one example, a conjugate of antithrombin and heparin where at least 50 wt % of the heparin is conjugated to antithrombin can be present. Furthermore, in one example, at least 98 wt % of the heparin in the composition has a molecular weight greater than 3,000 Daltons.

### PRIORITY INFORMATION

This application claims the benefit of U.S. Provisional Application No. 62/148,112, filed Apr. 15, 2015, which is incorporated herein by reference.

### BACKGROUND

Heparin is a sulfated polysaccharide which consists largely of an alternating sequence of hexuronic acid and 2-amino-2-deoxy-D-glucose. Heparin and a related compound, dermatan sulfate, are of great importance as anticoagulants for clinical use in the prevention of thrombosis and related diseases. They are members of the family of glycosaminoglycans, (GAGs), which are linear chains of sulfated repeating disaccharide units containing a hexosamine and a uronic acid. Anticoagulation using GAGs (such as heparin and dermatan sulfate) proceeds via their catalysis of inhibition of coagulant enzymes (the critical one being thrombin) by serine protease inhibitors (serpins) such as antithrombin III (referred to herein as simply “antithrombin” or “AT”) and heparin cofactor II (HCII). Binding of the serpins by the catalysts is critical for their action and occurs through specific sequences along the linear carbohydrate chain of the glycosaminoglycan (GAG). Heparin acts by binding to AT via a pentasaccharide sequence, thus potentiating inhibition of a variety of coagulant enzymes (in the case of thrombin, heparin also binds to the enzyme). Heparin can also potentiate inhibition of thrombin by binding to the serpin HCII. Dermatan sulfate acts by specifically binding to HCII via a hexasaccharide sequence, thus potentiating only the inhibition of thrombin. Since glycosaminoglycans (particularly heparin) can bind to other molecules in vivo or be lost from the site of action due to a variety of mechanisms, it would be advantageous to keep the GAG permanently associated with the serpin by a covalent bond. In further detail, it would be desirable to provide covalent conjugates of heparin and related glycosaminoglycans which retain high biological activity (e.g., anticoagulant activity) and improved pharmacokinetic properties and simple methods for their preparation.

### BRIEF DESCRIPTION OF THE DRAWINGS

FIG. 1 is a graph of absorbance (A₂₁₅ vs. H₂O) of each fraction of heparin eluted from a Sephadex® G-200 chromatography column.

FIG. 2 is a graph of absorbance of fractions of AT conjugated with heparin and fractions of AT alone eluted from a Sephadex® G-200 chromatography column.

FIG. 3 is a graph of absorbance (A₄₀₅ vs. H₂O) of reaction mixtures of four different reactions investigating a covalent antithrombin-heparin complex.

FIG. 4 is a graph of absorbance (A₄₀₅ vs. H₂O) of reaction mixtures of three different reactions investigating a covalent antithrombin-heparin complex that had been lyophilized from a high (concentrated) salt solution.

It should be noted that the figures are merely exemplary of several embodiments and no limitations on the scope of the present technology are intended thereby.

### DETAILED DESCRIPTION

Reference will now be made to exemplary embodiments and specific language will be used herein to describe the same. It will nevertheless be understood that no limitation of the scope of the disclosure is thereby intended. Alterations and further modifications of the inventive features described herein, and additional applications of the principles of the technology as described herein, which would occur to one skilled in the relevant art and having possession of this disclosure, are to be considered within the scope of the disclosure. Further, before particular embodiments are disclosed and described, it is to be understood that this disclosure is not limited to the particular process and materials disclosed herein as such may vary to some degree. It is also to be understood that the terminology used herein is used for the purpose of describing particular embodiments only and is not intended to be limiting, as the scope of the present disclosure will be defined only by the appended claims and equivalents thereof.

In describing and claiming the present technology, the following terminology will be used.

The singular forms “a,” “an,” and “the” include plural references unless the context clearly dictates otherwise. Thus, for example, reference to “an additive” includes reference to one or more of such components, “a solution” includes reference to one or more of such materials, and “a mixing step” refers to one or more of such steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, “hexose” refers to a carbohydrate (C₆H₁₂O₆) with six carbon atoms. Hexoses may be aldohexoses such as, for example, glucose, mannose, galactose, idose, gulose, talose, allose and altrose, whose open chain form contains an aldehyde group. Alternatively, hexoses may be ketoses such as fructose, sorbose, allulose and tagatose, whose open chain form contains a ketone group.

As used herein, “uronic acid” refers to the carboxylic acid formed by oxidation of the primary hydroxyl group of a carbohydrate and are typically named after the carbohydrate from which they are derived. Therefore, oxidation of the C6 hydroxyl of glucose gives glucuronic acid, oxidation of the C6 hydroxyl of galactose gives galacturonic acid and oxidation of the C6 hydroxyl of idose gives iduronic acid.

As used herein, “hexosamine” refers to a hexose derivative in which at least one hydroxy group, typically the C2 hydroxy group, has been replaced by an amine. The amine may be optionally alkylated, acylated (such as with muramic acid), typically by an acetyl group, sulfonated, (O or N-sulfated), sulfonylated, phosphorylated, phosphonylated and the like. Representative examples of hexosamines include glucosamine, galactosamine, tagatosamine, fructosamine, their modified analogs and the like.

As used herein, “glycosaminoglycan” refers to linear chains of largely repeating disaccharide units containing a hexosamine and a uronic acid. The precise identity of the hexosamine and uronic acid may vary widely and representative examples of each are provided in the definitions above. The disaccharide may be optionally modified by alkylation, acylation, sulfonation (O- or N-sulfated), sulfonylation, phosphorylation, phosphonylation and the like. The degree of such modification can vary and may be on a hydroxy group or an amino group. Most usually, the C6 hydroxyl and the C2 amine are sulfated. The length of the chain may vary and the glycosaminoglycan may have a molecular weight of greater than 200,000 Daltons, typically up to 100,000 Daltons, and more typically less than 50,000 Daltons. Glycosaminoglycans are typically found as mucopolysaccharides. Representative examples include, heparin, dermatan sulfate, heparan sulfate, chondroitin-6-sulfate, chondroitin-4-sulfate, keratan sulfate, chondroitin, hyaluronic acid, polymers containing N-acetyl monosaccharides (such as N-acetyl neuraminic acid, N-acetyl glucosamine, N-acetyl galactosamine, and N-acetyl muramic acid) and the like and gums such as gum arabic, gum Tragacanth and the like.

As used herein, “protein” includes, but is not limited to, albumins, globulins (e.g., immunoglobulins), histones, lectins, protamines, prolamines, glutelins, phospholipases, antibiotic proteins and scleroproteins, as well as conjugated proteins such as phosphoproteins, chromoproteins, lipoproteins, glycoproteins, nucleoproteins.

As used herein, “serpin” refers to a serine protease inhibitor and is exemplified by species such as antithrombin and heparin cofactor II.

As used herein, “amine” refers to primary amines, RNH₂, secondary amines, RNH(R′), and tertiary amines, RN(R′)(R″).

As used herein, “amino” refers to the group NH or NH₂.

As used herein, “imine” refers to the group C═N and salts thereof.

As used herein, the terms “treatment” or “treating” of a condition and/or a disease in a mammal, means: preventing the condition or disease, that is, avoiding any clinical symptoms of the disease; inhibiting the condition or disease, that is, arresting the development or progression of clinical symptoms; and/or relieving the condition or disease, that is, causing the regression of clinical symptoms. Treatment also includes use of the compositions of the present disclosure associated with a medical procedure with administration before, during or after the medical procedure.

In accordance with this, the present disclosure is drawn to compositions and methods for preparing heparin and antithrombin conjugates, as well as treating subjects with the compositions of the present disclosure. In one example, a composition for preventing thrombogenesis can comprise antithrombin and heparin, wherein at least 50 wt % of the heparin is conjugated to antithrombin, and wherein at least 98 wt % of the heparin in the composition has a molecular weight greater than 3,000 Daltons.

In another example, a method of making a composition for preventing thrombogenesis can comprise steps of conjugating antithrombin with heparin outside a body of a subject to form an antithrombin-heparin conjugate; preparing the antithrombin-heparin conjugate in a solution; and lyophilizing the antithrombin-heparin conjugate. In this example, the antithrombin-heparin conjugate can be in a solution of only water, water and from 0.01-0.09 molar alanine, or water and mannitol, for example.

In another example, a composition for preventing thrombogenesis can include an aqueous solution of antithrombin-heparin conjugate, wherein the antithrombin-heparin conjugate is present at a concentration of 9-11 mg/mL with respect to the entire volume of the solution. The antithrombin-heparin conjugate can be formed by conjugating antithrombin with heparin outside a body of a subject.

In yet another example, a method of making a composition for preventing thrombogenesis can include antithrombin with heparin outside a body of a subject to form an antithrombin-heparin conjugate, wherein the yield of the antithrombin-heparin conjugate is defined such that at least 60 wt % of the starting antithrombin taken for reaction and becomes conjugated to heparin (i.e. used to make the antithrombin-heparin conjugate).

In still another example, a composition for preventing thrombogenesis can include antithrombin, heparin, and antithrombin-heparin conjugate, wherein the antithrombin-heparin conjugate is present at a yield of at least 60 wt % of the starting antithrombin used to make the antithrombin-heparin conjugate.

In another example, a method of treating a condition or disease can include administering an antithrombin-heparin conjugate prepared in accordance with examples of the present technology to a mammal in need thereof. In further detail, these treatments can be carried about by administering the heparin and antithrombin conjugates of the present disclosure to a subject, such as a human, in need of such a treatment. Conditions and diseases that can be treated using the conjugate compositions described herein include myocardial infarction and a large array of thrombotic states. These include fibrin deposition found in neonatal respiratory distress syndrome, adult respiratory distress syndrome, primary carcinoma of the lung, non-Hodgkins lymphoma, fibrosing alveolitis, and lung transplants, to name a few. Also, the present compositions can treat either acquired AT deficient states such as neonatal respiratory distress syndrome, L-asparaginase induced deficiency, cardiopulmonary bypass induced deficiency, sepsis or congenital AT deficient states. In the case of congenital AT deficiency, life threatening thrombotic complications with AT levels of less than 0.25 Units/ml in heterozygotes requiring AT plus heparin may occur in up to 1 or 2 infants per year in the U.S.A. The conditions and diseases treated in the present disclosure include those characterized by excess thrombin generation or activity. Such conditions often occur where a subject has been exposed to trauma, for example in surgical patients. Trauma caused by wounds or surgery results in vascular damage and secondary activation of blood coagulation. These undesirable effects may occur after general or orthopedic surgery, gynecologic surgery, heart or vascular surgery, or other surgical procedures. Excess thrombin may also complicate progression of natural diseases such as artherosclerosis which can cause heart attacks, strokes or gangrene of the limbs. Therefore, the methods and compositions of the present technology can be used to treat, prevent, or inhibit a number of important cardiovascular complications, including unstable angina, acute myocardial infarction (heart attack), cerebral vascular incidents (stroke), pulmonary embolism, deep vein thrombosis, arterial thrombosis, etc. The compositions and methods of the technology may be used to reduce or prevent clotting during dialysis and reduce or prevent intravascular coagulation during open heart surgical procedures. In additional detail, in aspects of the disclosure, methods and compositions are provided for preventing or inhibiting thrombin generation or activity in patients at increased risk of developing a thrombus due to medical conditions that disrupt hemostasis (e.g., coronary artery disease, atherosclerosis, etc.). In another aspect, methods and compositions are provided for patients at increased risk of developing a thrombus after a medical procedure, such as cardiac surgery, vascular surgery, or percutaneous coronary interventions. In an embodiment, the methods and compositions of this disclosure are used in cardiopulmonary bypass surgery. The compositions can be administered before, during or after the medical procedure.

Turning now to various embodiments and details related to the present disclosure, it is known that heparin is readily available in an unfractionated form, which contains molecules with a wide range of molecular weights. By removing from most to all of the heparin molecules having molecular weights less than 3,000 Daltons prior to conjugating the heparin with the antithrombin, the activity of the antithrombin-heparin conjugate can be enhanced. In an additional embodiment, heparin molecules having a molecular weight less than 5,000 Daltons can be from mostly to completely removed.

The antithrombin-heparin conjugates formed using heparin from which low molecular weight heparin molecules have been removed are compositionally different from other antithrombin-heparin conjugates. Low molecular weight heparin chains can be removed from the heparin prior to reaction with AT to synthesize the antithrombin-heparin conjugate (ATH). Therefore, the ATH is devoid of low molecular weight heparin chains conjugated to the AT.

Low molecular weight heparin chains can be removed from commercially available heparin prior to reacting the heparin with AT to form ATH. This produces ATH that is compositionally different from ATH formed from unfractionated heparin without removing the low molecular weight heparin before reaction with AT. Additionally, forming ATH from unfractionated heparin and then subsequently removing low molecular weight ATH does not produce the same product as the ATH of the present disclosure. Without being bound to any particular theory, it is believed that low molecular weight heparin chains (such as less than 3,000 Daltons or less than 5,000 Daltons) compete with longer chain heparins for conjugating to AT. The very low molecular weight heparin chains have a high proportion of aldose termini which react with the AT. Therefore, the very low molecular weight heparin chains tend to conjugate with AT more quickly, out-competing the higher molecular weight heparin chains. However, once the very low molecular weight heparin chains are bonded to the AT, the chains do not contain sufficient sites or length for binding thrombin and Factor Xa, an enzyme involved in the coagulation cascade. The inhibitory activity against factor Xa and thrombin drops dramatically in the lowest molecular weight range of heparin molecules. Thus, the ATH formed from these very low molecular weight heparin chains has essentially zero activity for preventing thrombogenesis. Although commercial heparin contains a relatively small percentage of heparin chains below 5,000 Daltons, these very low molecular weight heparin chains have such a high reactivity with AT that a significant amount of the ATH formed contains the very low molecular weight heparin chains.

If the very low molecular weight heparin is not removed first, prior to conjugation, then a greater proportion of reactive termini in this population versus that of the higher molecular weight heparin will tend to outcompete the other heparin molecules to a varying degree across the entire molecular weight spectrum (as the proportion of aldose termini varies continually across the whole molecular weight range of heparin). This can have adverse effects on the final ATH. First, the ATH will contain a significant population of ATH molecules containing very small heparin chains with no activity. Second, the remaining ATH molecules (outside of this very low molecular weight range of ATH) will contain a population of heparin that has a reduced proportion of heparin chains in discrete molecular weight ranges that had fewer aldose termini to compete with the inactive low molecular weight heparin chains. This low aldose type heparin tends to be in the much longer chains but is not entirely defined by a straight relationship between heparin chain length and aldose termini required for linkage to AT.

Furthermore, heparin with at least 18 monosaccharide units can also be more effective at inhibiting thrombin. At least 18 monosaccharide units are used to bind both antithrombin and thrombin. The mechanism by which heparin binds antithrombin and thrombin is referred to as the template or bridging mechanism. Heparin can exert its effect via conformational activation by binding to AT and allosterically converting the AT into a structural form that is much more reactive towards coagulation proteases. Alternatively, heparin may act as a template through binding to both inhibitor and enzyme, thus localizing the molecules for reaction. In this mechanism, conformational activation of AT by heparin occurs but additional reaction rate enhancement is gained by simultaneous binding of heparin to the enzyme, thus assisting approach of the coagulation factor towards the activated inhibitor. The particular minimum chain length of 18 monosaccharides may explain why there is a very abrupt drop in activity against thrombin within the low molecular weight fraction of heparin. From the structure for a monosulfated uronic acid-disulfated glucosamine heparin disaccharide, that is without the sodium or other ions found in a salt form, the MW of an 18 saccharide (9 disaccharide) chain would be about 4500 Daltons.

Somewhat lower molecular weight heparin chains may be useful for inhibiting Factor Xa. A particular pentasaccharide sequence in heparin can bind to AT and activate the AT for inhibiting Factor Xa. The particular pentasaccharide sequence has been made available on its own as the pharmaceutical “Fondaparinux,” but the sequence can occur in heparin chains as well. The sequence of monosaccharides is shown in Formula I:

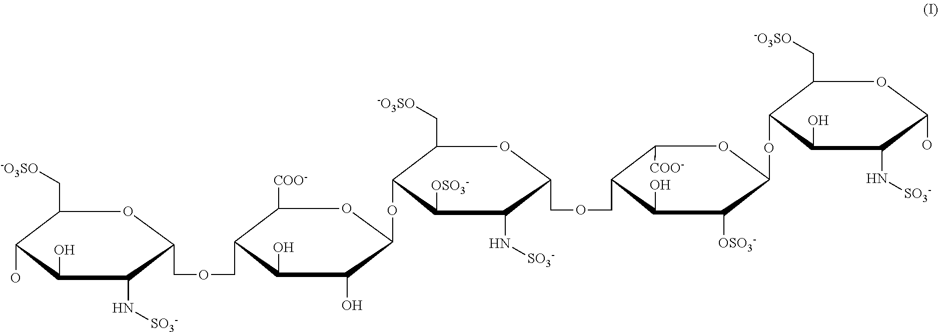

Thus, heparin chains with less than 18 monosaccharides that contain this pentasaccharide sequence may be able to activate AT to inhibit Factor Xa even though the chains are not long enough to bind to AT and thrombin.

The longest heparin chains can in some case have the highest inhibitory activity. However, some mid-range and lower molecular weight heparin chains can have significantly less undesirable binding to other plasma proteins and platelets. Therefore, these mid-range heparin chains can be more selective for inhibiting thrombin and factor Xa without causing unwanted side effects such as platelet dysfunction from binding with platelets and binding other materials.

Isolating the higher molecular weight ATH after the conjugation to give very long chain ATH provides a less desirable and distinct product compared to the present technology which separates out (substantially or fully) the heparin prior to conjugation. For example, the proportion of 2-pentasaccharide high activity molecules in this subpopulation may be altered because of a differential ability of these high activity chains to compete with the very low molecular weight heparins for conjugation. Additionally, isolating the high molecular weight ATH after conjugation eliminates ATH molecules with mid-range and lower sized heparin chains that are also active and have other desirable characteristics such as reduced non-selective binding to plasma proteins and platelets.

Alternatively, attempts to react all aldose-terminating heparin chains with AT by increasing the ratio of AT to heparin in the reaction mixture are not likely to succeed because many experiments have shown that only up to 60 wt % conversion of AT into ATH is obtained even with the aldose containing heparin in several-fold excess and at highest practical concentrations. Reducing the proportion of heparin to AT even more will only decrease the ATH yield further without any promise that all of the active longer chains will be incorporated into the product.

In some embodiments, a composition for preventing thrombogenesis can contain ATH formed from commercial heparin from which substantially all of the heparin chains with a molecular weight less than 3,000 Daltons have been removed (e.g., at least 98 wt % of remaining heparin chains can have a molecular weight greater than 3,000 Daltons). In other embodiments, heparin chains with a molecular weight less than 5,000 Daltons can be substantially removed or removed. Thus, the ATH product can contain heparin chains that range in molecular weight from 3,000 Daltons (or 5,000 Daltons) up to the highest molecular weights contained in the commercial heparin. In certain examples this range of molecular weights can be from 3,000 Daltons to 50,000 Daltons, or from 5,000 Daltons to 50,000 Daltons. In additional examples, at least a portion of the heparin chains can be in a mid-molecular weight range. For example, at least a portion of the heparin chains in the ATH can have a molecular weight from 3,000 Daltons to 30,000 Daltons, from 3,000 Daltons to 20,000 Daltons, from 3,000 Daltons to 15,000 Daltons, from 3,000 Daltons to 10,000 Daltons, from 5,000 Daltons to 30,000 Daltons, from 5,000 Daltons to 20,000 Daltons, from 5,000 Daltons to 15,000 Daltons, or from 5,000 Daltons to 10,000 Daltons. Thus, the ATH can be substantially devoid or devoid of heparin chains with a molecular weight below 3,000 Daltons or 5,000 Daltons.

Commercial heparin can typically contain a range of heparin chains with molecular weights ranging from 1,000 Daltons or less to 50,000 Daltons or more. The lowest molecular weight fraction, such as the chains with molecular weights below 3,000 or 5,000 Daltons, can be removed by any suitable method. Non-limiting examples of methods for removing the low molecular weight chains include dialysis, diafiltration, gel filtration and electrophoresis. Dialysis or diafiltration can be performed under high salt conditions. For example, high salt conditions for dialysis or diafiltration can include salt concentrations from about 1 M NaCl to about 4 M NaCl. Salts other than NaCl can also be used. The high salt concentration can assist movement of the small chains through membranes having appropriate pore sizes. Gel filtration can be performed using a suitable media for separating molecules by size. In one particular example, gel filtration can be performed on Sephadex® G-200, which is a gel media for separating molecules with molecular weights in the range of 1,000 to 200,000 Daltons. Commercial heparin can be gel filtered on a column of gel media, and a series of fractions can be eluted with the first fractions containing the highest molecular weight chains and the subsequent fractions containing progressively lower molecular weights. The molecular weights of heparin in each fraction can be determined, and the fractions having the desired molecular weights can be pooled. Using this method, fractions containing heparin with molecular weights below the threshold of 3,000 or 5,000 Daltons can be excluded. If desired, heparin chains above a certain threshold can also be excluded. For example, fractions containing heparin above 50,000 Daltons, 30,000 Daltons, 20,000 Daltons, 15,000 Daltons, or 10,000 Daltons can be excluded if desired. The pooled fractions having the desired range of molecular weights can then be used to synthesize ATH.

It should be noted that the methods of removing the very low molecular weight heparin chains described above are only exemplary and should not be considered limiting. Any method of processing commercial heparin to remove heparin chains below a certain threshold molecular weight can be used in the present disclosure.

ATH can be formed by conjugating AT with the heparin that is now devoid of very low molecular weight chains. Exemplary methods of conjugating heparin with AT are disclosed in U.S. Pat. No. 7,045,585, which is incorporated herein by reference. These methods can be applied to forming ATH using heparin from which the very low molecular weight chains have been removed, as described herein. Heparin can be conjugated with AT through a simple one-step process, which provides for direct covalent attachment of the amine of an amine containing moiety (such as, but not limited to, amine containing oligo(poly)saccharides, amine containing lipids, proteins, nucleic acids and any amine containing xenobiotics) to a terminal aldose residue of a heparin chain. For forming ATH, the amine containing moiety is present in the AT, although other proteins can be conjugated using the same methods. The mild non-destructive methods provided herein allow for maximal retention of biological activity of the protein and allow direct linkage of the protein without the need for intermediate spacer groups.

In one embodiment, heparin is incubated with AT at a pH suitable for imine formation between the amine and the terminal aldose or ketose residue of the heparin. Terminal aldose and ketose residues generally exist as an equilibrium between the ring closed cyclic hemiacetal or hemiketal form and the corresponding ring opened aldehyde or ketone equivalents. Generally, amines are capable of reacting with the ring opened form to produce an imine (Schiff base). Typically, the aldoses are more reactive because the corresponding aldehydes of the ring open form are more reactive towards amines. Therefore, covalent conjugate formation between amines and terminal aldose residues of heparin provides a method of attaching the AT containing an amine to the heparin.

The reaction is typically carried out at a pH of about 4.5 to about 9, and more typically at about 5 to about 8, and even more typically about 7 to about 8. The reaction is generally done in aqueous media. However, organic media, especially polar hydrophilic organic solvents such as alcohols, ethers and formamides and the like may be employed in proportions of up to about 40% to increase solubility or reactivity of the reactants, if necessary. Non-nucleophilic buffers such as phosphate, acetate, bicarbonate and the like may also be employed.

In some cases the imines formed by condensation of the amines of the AT with the terminal aldose residues of the heparin are reduced to the corresponding amines. This reduction may be accomplished concurrently with imine formation or subsequently. A wide array of reducing agents may be used, such as hydride reducing agents including sodium borohydride or sodium cyanoborohydride. In one example, any reducing agent that does not reduce disulfide bonds can be used.

Alternatively, if reduction of the intermediate imine is not desired, the imine may be incubated for a sufficient period of time, typically about 1 day to 1 month, more typically about 3 days to 2 weeks, to allow Amadori rearrangement of the intermediate imine. The terminal aldose residues of the heparins conjugated by the methods provided by this disclosure frequently possess C2 hydroxy groups on the terminal aldose residue, i.e., a 2-hydroxy carbonyl moiety which is converted to a 2-hydroxy imine by condensation with the amine of the AT being conjugated to the heparin. In the Amadori rearrangement, which is particularly common in carbohydrates, the α-hydroxy imine (imine at C1, hydroxy at C2) formed by the initial condensation may rearrange to form an (α-keto amine by enolization and re-protonation (keto at C2, amine at C1)). The resulting α-carbonyl amine is thermodynamically favored over the precursor α-hydroxy imine, thus providing a stable adduct with minimal disruption of the heparin chain. Thus, in this embodiment, the technology provides a heparin chain covalently conjugated at the C1 of the terminal aldose residue of the heparin to an amine containing AT via an amine linkage. If desired, the resulting conjugate may be reduced or labelled by reduction of the C2 carbonyl group with a labelling reagent, such a radiolabel (e.g., NaB³H₄), or conjugated to a second amine containing species, such as a fluorescent label.

Although the above description focuses on heparin and AT, a variety of different amine containing species may be conjugated to a variety of glycosaminoglycans by the methods disclosed herein. The primary amine may be on a small molecule, such as, for example, a drug or fluorescent or chromophoric label or a macromolecule such as, for example, a protein (antibodies, enzymes, receptors, growth factors and the like), a polynucleotide (DNA, RNA and mixed polymers thereof) or a polysaccharide. Generally, when proteins are being conjugated to glycosaminoglycans, linkage will occur through the ε-amino groups of lysine residues. Alternatively, linkage may also be accomplished via the α-amino group of the N-terminal amino acid residue. In addition, many other methods can be used that are known to those of skill in the art to introduce an amine functionality into a macromolecule.

In particular, the present technology can be applied to a variety of other therapeutically useful proteins where longer half-life and blood coagulation considerations are important. These include blood enzymes, antibodies, hormones and the like as well as related plasminogen activators such as streptokinase and derivatives thereof. In particular, this technology provides conjugates of heparin or dermatan sulfate with antithrombin, heparin cofactor II (HCII) or analogs of heparin cofactor II.

The methods of the present disclosure provide glycosaminoglycan conjugates with maximal retention of biological activity. In particular, conjugates of heparin or dermatan sulfate with either AT or HCII are provided which possess >60 wt %, typically >90 wt %, more typically >95 wt %, and most typically >98 wt % of intact unconjugated heparin antithrombin activity. The methods of the present technology provide intact heparin molecules conjugated to antithrombin or heparin cofactor II. Thus, loss of biological activity associated with fragmentation or other modification of heparin prior to conjugation is avoided. The heparin conjugates of this technology retain their anticoagulant activity because of their preparation from intact heparin. Therefore, the methods disclosed herein can be used to prepare active heparin conjugates by first attaching linking groups and spacers to the species sought to be conjugated to heparin (or whatever the glycosaminoglycan being used) and subsequently attaching it to heparin. Numerous methods of incorporating reactive amino groups into other molecules and solid supports are described in the InmunoTechnology Catalog and Handbook, Pierce Chemical Company (1990), incorporated by reference. Thereby, any species possessing reactive amino groups or capable of being modified to contain such amino groups, by any method presently known or that becomes known in the future, may be covalently conjugated to glycosaminoglycans, such as heparin, by the methods disclosed herein and all such conjugates are contemplated by this disclosure.

As described above, the present technology takes advantage of the fact that native (isolated from intestinal mucosa) heparin, as well as dermatan sulfate, already contains molecules with aldose termini which would exist in an equilibrium between hemiacetal and aldehyde forms. Thus, heparin or dermatan sulfate can be conjugated to antithrombin serpins by reduction of the single Schiff base formed spontaneously between the aldose terminus aldehyde on heparin or dermatan sulfate and an amino on the serpin. The heparin or dermatan sulfate is unmodified (unreduced in activities) prior to conjugation and is linked at one specific site at one end of the molecule without any unblocked activation groups or crosslinking of the serpin.

In another aspect of this disclosure, covalent complexes can be produced by simply mixing heparin and AT in buffer and allowing a keto-amine to spontaneously form by an Amadori rearrangement between the heparin aldose terminus and an AT amino group. Thus, this technology provides methods of using the Amadori rearrangement to prepare conjugates of glycosaminoglycans to amine containing species, particularly proteins. This is a particularly mild and simple method of conjugation, which minimizes the modification of the glycosaminoglycan, thus maximizing the retention of its biological activity.

Another aspect of this technology provides covalent conjugates of glycosaminoglycans, particularly of heparin, end-labelled with an amine containing species at the terminal aldose residue of the glycosaminoglycan. For example, heparin and AT can be linked directly together so that the active pentasaccharide sequence for AT on the heparin is in close proximity for binding. This is one of the fundamental reasons for making a covalent heparin-AT complex, as heparin accelerates inhibition through AT only if AT can bind the active sequence. It is notable that ATH has the unique property that the H (heparin) in the conjugate stoichiometrically activates the endogenous AT while catalytically activating exogenous AT. Typically, one amine containing species will be attached to each glycosaminoglycan. However, it will be apparent that the ratio of amine containing species to glycosaminoglycan may be reduced below one by adjusting the molar ratios of the reactants or the time of the reaction.

Glycosaminoglycans are available in a variety of forms and molecular weights. For example, heparin is a mucopolysaccharide, isolated from pig intestine or bovine lung and is heterogenous with respect to molecular size and chemical structure. It consists primarily of (1-4) linked 2-amino-2-dexoxy-α-D-gluopyranosyl, and α-L-idopyranosyluronic acid residues with a relatively small amount of β-D-glucopyranosyluronic acid residues. The hydroxyl and amine groups are derivatized to varying degrees by sulfation and acetylation.

Heparin molecules can also be classified on the basis of their pentasaccharide content. About one third of heparin contains chains with one copy of the unique pentasaccharide with high affinity for AT, whereas a much smaller proportion (estimated at about 1% of total heparin) consists of chains which contain more than one copy of the high affinity pentasaccharide. The remainder (approximately 66%) of the heparin does not contain the pentasaccharide. Thus, so called “standard heparin” constitutes a mixture of the three species, “low affinity” heparin that lacks a copy of the pentasaccharide, “high affinity” heparin that is enriched for species containing at least one copy of the pentasaccharide, and “very high affinity” heparin that refers to the approximately 1% of molecules that contain more than one copy of the pentasaccharide. These three species can be separated from each other using routine chromatographic methods, such as chromatography over an antithrombin affinity column.

One advantage of forming a conjugate between heparin and a species containing at least one primary amino group (e.g., AT) using the slow glycation process disclosed herein, is the apparent selection for heparin chains having two pentasaccharides. Thus, for example, ATH prepared by the method of this disclosure appears to be enriched for heparin species containing two pentasaccharides. When standard heparin (containing approximately 1% of two-pentasaccharide heparin) is used as a starting material, usually more than 10% of the resulting ATH comprises two-pentasaccharide heparin, more often more than about 20%, frequently more than 35%, and often more than about 50% of the ATH comprises two-pentasaccharide heparin.

This enrichment may account for several useful properties of ATH. The ATH of the present technology activates the AT to which it is conjugated, in a stoichiometric fashion, but activates exogenous AT in a catalytic fashion. Thus, the heparin within the ATH complex acts catalytically both when ATH is administered as systemic anticoagulant and when ATH is used to coat surfaces to render them non-thrombogenic. The method of the technology produces an ATH complex with very high specific anti-factor IIa activity. In addition, the second pentasaccharide chain in the ATH complex can interact with exogenous AT molecules, thereby allowing the conjugated heparin to have catalytic activity. Moreover, the heparin in the ATH complex can be orientated in such a way that the pentasaccharide is available to bind and activate circulating AT molecules when the ATH complex is bound to the prosthetic surface.

It will be appreciated that a heparin conjugate of interest (e.g., ATH) can also be produced by incubating a species containing at least one primary amino group (e.g., AT) with purified very high affinity heparin (i.e., containing two pentasaccharide groups) or a fraction enriched for very high affinity heparin.

Though this technology has been illustrated primarily with respect to heparin, it is apparent that all glycosaminoglycans, irrespective of their molecular weight and derivatization, may be conjugated by the methods disclosed herein, provided they possess a terminal aldose residue. Conjugates of all such glycosaminoglycans and their preparation by the methods herein are within the scope of this disclosure. For example, conjugates of heparin derivatized with phosphates, sulfonates and the like as well as glycosaminoglycans with molecular weights lower or higher than the molecular weights of heparin are within the scope of this disclosure.

In a further aspect of the present disclosure, a method of making a composition for preventing thrombogenesis can include conjugating AT with heparin outside a body of a subject to form an antithrombin-heparin conjugate, wherein the amount of antithrombin yielded in the antithrombin-heparin conjugate is greater than 60 wt %, greater than 65 wt %, greater than 75 wt %, greater than 85 wt %, greater than 90 wt %, greater than 95 wt %, or greater than 99 wt % based on the starting antithrombin used in the synthesis. The yield can be increased by various methods. In one example, AT can be conjugated to heparin by the methods described above. Following the conjugation, any unbound AT can be recycled and used in another conjugation reaction with heparin. After each step of incubating AT with heparin, the unbound AT can be recycled and used to make additional ATH.

In another example, the yield of ATH can be increased by using an Amadori rearrangement catalyst. Non-limiting examples of catalysts that can increase the rate of Amadori rearrangement include 2-hydroxypyridine, tertiary amine salts, ethyl malonate, phenylacetone, acetic acid, as well as other acids. In a particular example, AT and heparin can be reacted in the presence of 2-hydroxypyridine while being heated in water or very amphiphilic solvents such as formamide. In further examples, AT and heparin can be reacted in the presence of trimethylamine or trimethylamine salts.

The rate of the Amadori rearrangement can also be increased by Amadori rearrangement accelerating solvent systems. Non-limiting examples of solvents include mixtures of water with formamide, dimethylformamide, dioxane, ethanol, dimethylsulfoxide, pyridine, acetic acid, trimethylamine, triethylamine, and combinations thereof. Heparin and AT can be reacted in these solvent systems to accelerate the Amadori rearrangement to form ATH.

An additional method for increasing the rate of conjugating the heparin aldose to an amine-containing molecule involves using a linking agent. The linking agent can be a heterobifunctional agent, with a group reactive toward the aldose of heparin at one end and a different group at the other end that can be used for linking either to AT or to a secondary linking agent that can then be linked to AT. In one particular example, the linking agent can contain hydrazine at one end and an amino group at the other end, such as 2-aminoethylhydrazine. This linking agent can be reacted with heparin to form a hydrazine with the aldose aldehyde of the heparin. The product can be dialyzed or diafiltered with membranes that allow heparin chains less than 3,000 or 5,000 Daltons in molecular weight to be removed along with any unreacted linking agent. The heparin-hydrazone product can then be reacted with a large excess of a secondary linking agent. The secondary linking agent can be a homobifunctional reagent possessing activated carboxyl groups at each end, such as succinic acid di(N-hydroxysuccinimide) ester (prepared by esterifying succinic acid with N-hydroxysuccinimide using condensing agents such as carbonyldiimidazole or a carbodiimide) so that the amino group on the hydrazine linking agent reacts with just one of the activated carboxyls on the secondary linking agent. The reaction mixture can be dialyzed or diafiltered to remove unreacted secondary linking agent. At this point, the product is heparin modified with the amino-hydrazine linking agent as well as the secondary linking agent. This product can be incubated with AT in buffered H₂O so that the amino group on the AT reacts with the second activated carboxyl group on the secondary linking agent to form an AT-Heparin conjugate, where the AT and heparin are linked by the linking agent and the secondary linking agent.

After forming ATH, the ATH can be lyophilized (freeze-dried) for storage. In one embodiment, the ATH can be prepared in a solution containing only water and then lyophilized. In another embodiment, the ATH can be prepared in a solution with water and alanine at a concentration of from 0.01-0.09 molar, and then lyophilized. In yet another embodiment, the ATH can be prepared in a solution containing water and mannitol, and then lyophilized. Each of these methods can be used independently, and each method can provide its own advantages. After lyophilization using any of these methods, the ATH can be reconstituted and retain a significant amount of its activity for inhibiting thrombin compared to its activity prior to lyophilization. In some cases, the ATH can retain at least 50%, at least 60%, at least 70%, at least 80%, at least 90%, at least 95%, or at least 98% of its activity for inhibiting thrombin. It has been found that using other methods of lyophilizing ATH, such as preparing the ATH in a solution containing greater than 1 molar salt before lyophilization, can destroy the activity of the ATH.

Whether the ATH has been lyophilized or not, the ATH can be prepared in an aqueous solution containing from 9-11 mg/mL of ATH with respect to the entire volume of the solution. It has been found that forming solutions with an ATH concentration higher than 11 mg/mL can lead to aggregation of ATH that is difficult or impossible to reverse. However, stable aqueous solutions can be prepared with ATH concentrations of 9-11 mg/ml. This solution can be formulated for administration to a subject for treatment of any of the conditions described herein. The solution can also include a variety of additives as are suitable for administering to a subject.

In clinical practice, the heparin conjugates of the present technology may be used generally in the same manner and in the same form of pharmaceutical preparation as commercially available heparin for clinical use. Thus, the heparin conjugates provided by the present technology may be incorporated into aqueous solutions for injection (intravenous, subcutaneous and the like) or intravenous infusion or into ointment preparations for administration via the skin and mucous membranes. Any form of therapy, both prophylactic and curative, either currently known or available in the future, for which heparin therapy is indicated may be practiced with the heparin conjugates provided by this technology.

The heparin conjugates of this technology find particular utility in the treatment of neonatal and adult respiratory distress syndrome (RDS). In contrast to the use of noncovalent heparin-AT complexes, the use of the covalent heparin conjugates of the present technology prevents loss of heparin in the lung space by dissociation from AT. In this case, a solution of covalent complex in a physiologic buffer can be delivered as an atomized spray down the airway into the lung via a catheter or puffer. Due to its large size, ATH will remain in the alveoli for a longer period of time. ATH is also useful for treatment of idiopathic pulmonary fibrosis.

Long term use in the circulation can be carried out by either intravenous or subcutaneous injection of the complex in a physiologic buffer. The covalent conjugates of this technology may also be used in the treatment of acquired AT deficient states characterized by thrombotic complications such as cardiopulmonary bypass, extracorporeal molecular oxygenation, etc. because a longer half-life of the covalent complex allows for fewer treatments and less monitoring. Additionally, this disclosure provides for prophylactic treatment of adult patients at risk for deep vein thrombosis.

The ATH conjugate of this technology has numerous advantages over uncomplexed AT and standard heparin. Since the AT is covalently linked to the heparin, non-specific binding of ATH to plasma proteins will be less than occurs with standard heparin, resulting in less inter-individual variation in dose response to ATH than there is to standard heparin. The longer half-life of ATH after intravenous injection in humans means that a sustained anticoagulant effect may be obtained by administering ATH less frequently than is required for uncomplexed AT and standard heparin. ATH is a much more effective inactivator of thrombin and factor Xa than AT, and can be effective when used in much lower concentrations than AT in patients with AT deficiency. In addition, ATH can access and inhibit thrombin bound to fibrin. Finally, when linked (e.g., covalently linked) to prosthetic surfaces (e.g., endovascular grafts), ATH has shown much greater antithrombotic activity in vivo than covalently linked AT, covalently linked heparin, or covalently linked hirudin.

Premature infants have a high incidence of respiratory distress syndrome (RDS), a severe lung disease requiring treatment with assisted ventilation. Long term assisted ventilation leads to the onset of bronchopulmonary dysplasia (BPD) as a result of lung injury which allows plasma coagulation proteins to move into the alveolar spaces of the lung. This results in the generation of thrombin and subsequently fibrin. The widespread presence of fibrin within the lung tissue and airspaces is consistently observed in infants dying of RDS. This fibrin gel within the airspace impairs fluid transport out of the lung airspaces resulting in persistent and worsening pulmonary edema. The present technology provides for treatment of such fibrin-mediated diseases in lung tissue by preventing intra-alveolar fibrin formation by maintaining an “anti-thrombotic environment” and/or enhancing fibrinolysis within lung tissue, thereby decreasing the fibrin load in the air spaces of the lung.

The heparin conjugates can be delivered directly to the airspaces of the lung via the airway prophylactically (before the baby takes its first breath). This ensures that the antithrombotic agent is available directly at the site of potential fibrin deposition and that the bleeding risk associated with systemic antithrombotic therapies is avoided. In addition, the antithrombotic agent will already be present in the lung prior to the start of the ventilatory support which is associated with the initial injury, i.e., unlike systemic antithrombin administration where crossing of the administered drug to the lung airspace does not occur until after lung injury. Since heparin is covalently attached to AT it will remain in the lung airspaces. It can also be an adjunctive therapy to the surfactants currently administered to prevent RDS and BPD. By “lung surfactant” is meant the soap-like substance normally present in the lung's airspaces whose main role is to prevent collapse of the airspace, as well as assist gas transfer. The conjugates can also be delivered repeatedly via the endotracheal tube or as an inhaled aerosol. Adjunctive therapy can also be practiced with asthma medications by inhaler (e.g., anti-inflammatory steroids such as beclomethasone dipropionate), other anti-asthmatics such as cromolyn sodium (disodium salt of 1,3-bis(2-carboxychromon-5-yloxy)-2-hydroxypropane, (“INTAL”) and bronchodilators such as albuterol sulfate.

A variety of other diseases associated with elevated thrombin activity and/or fibrin deposition can be treated by administration of the conjugates of this disclosure. The inflammatory processes involved in adult respiratory distress syndrome are fundamentally similar to neonatal RDS and can be treated by the antithrombotic therapy described. Spontaneous lung fibrosis has also been shown to have activation of the coagulation/fibrinolytic cascades in the lung airspaces. Fibrotic disease of the lung is often a side effect associated with cancer chemotherapy and the RDS antithrombotic administration of the covalent heparin conjugates of this technology can be administered prophylactically prior to cancer chemotherapy to prevent lung fibrosis. Administration is repeated after chemotherapy in order to ensure no fibrin formation. A decrease in antithrombin activity and an increase in thrombin activity in sepsis is also well documented. Sepsis is the most common risk factor for developing adult RDS. Thus, the heparin conjugates of this disclosure can be used to reduce the mortality associated with septic shock.

The conjugates of this disclosure can be administered at a therapeutically effective dosage, i.e., that amount which, when administered to a mammal in need thereof, is sufficient to effect treatment, as described above (for example, to reduce or otherwise treat thrombosis in the mammal, or to inactivate clot-bound thrombin, or to inhibit thrombus accretion). Administration of the active compounds and salts described herein can be via any of the accepted modes of administration for agents that serve similar utilities.

Generally, an acceptable daily dose is of about 0.001 to 50 mg per kilogram body weight of the recipient per day, about 0.05 to 25 mg per kilogram body weight per day, or about 0.01 to 10 mg per kilogram body weight per day. Thus, for administration to a 70 kg person, the dosage range can be about 0.07 mg to 3.5 g per day, about 3.5 mg to 1.75 g per day, or about 0.7 mg to 0.7 g per day depending upon the individuals and disease state being treated. In the case of ATH, the long half-life allows the compound to be administered less frequently than standard heparin (e.g., once or twice weekly).

Administration can be via any accepted systemic or local route, for example, via parenteral, intravenous, nasal, bronchial inhalation (i.e., aerosol formulation), transdermal or topical routes, in the form of solid, semi-solid or liquid dosage forms, such as for example, tablets, suppositories, pills, capsules, powders, solutions, suspensions, aerosols, emulsions or the like, such as in unit dosage forms suitable for simple administration of precise dosages. Usually, aqueous formulations can be used. The conjugate can be formulated in a non-toxic, inert, pharmaceutically acceptable carrier medium, at a pH of about 3-8 or at a pH of about 6-8. Generally, the aqueous formulation can be compatible with the culture or perfusion medium. The compositions will include a conventional pharmaceutical carrier or excipient and a conjugate of the glycosaminoglycan, and in addition, may include other medicinal agents, pharmaceutical agents, carriers, adjuvants, etc. Carriers can be selected from the various oils, including those of petroleum, animal, vegetable or synthetic origin, for example, peanut oil, soybean oil, mineral oil, sesame oil, and the like. Water, saline, aqueous dextrose or mannitol, and glycols are examples of suitable liquid carriers, particularly for injectable solutions. Suitable pharmaceutical carriers include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. Other suitable pharmaceutical carriers and their formulations are described in Remington's Pharmaceutical Sciences by E. W. Martin (1985).

If desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, etc.

The compounds of this disclosure can be administered as a pharmaceutical composition which comprises a pharmaceutical excipient in combination with a conjugate of the glycosaminoglycan. The level of the conjugate in a formulation can vary within the full range employed by those skilled in the art, e.g., from about 0.01 percent weight (% w) to about 99.99% w of the drug based on the total formulation and about 0.01% w to 99.99% w excipient. In one example, the formulation can be about 3.5 to 60% by weight of the pharmaceutically active compound, with the rest being suitable pharmaceutical excipients.

### EXAMPLES

The following examples illustrate embodiments of the disclosure that are presently best known. However, it is to be understood that the following are only exemplary or illustrative of the application of the principles of the present technology. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following examples provide further detail in connection with what are presently deemed to be practical embodiments of the disclosure.

### Example 1

### Removing Very Low Molecular Weight Heparin Chains

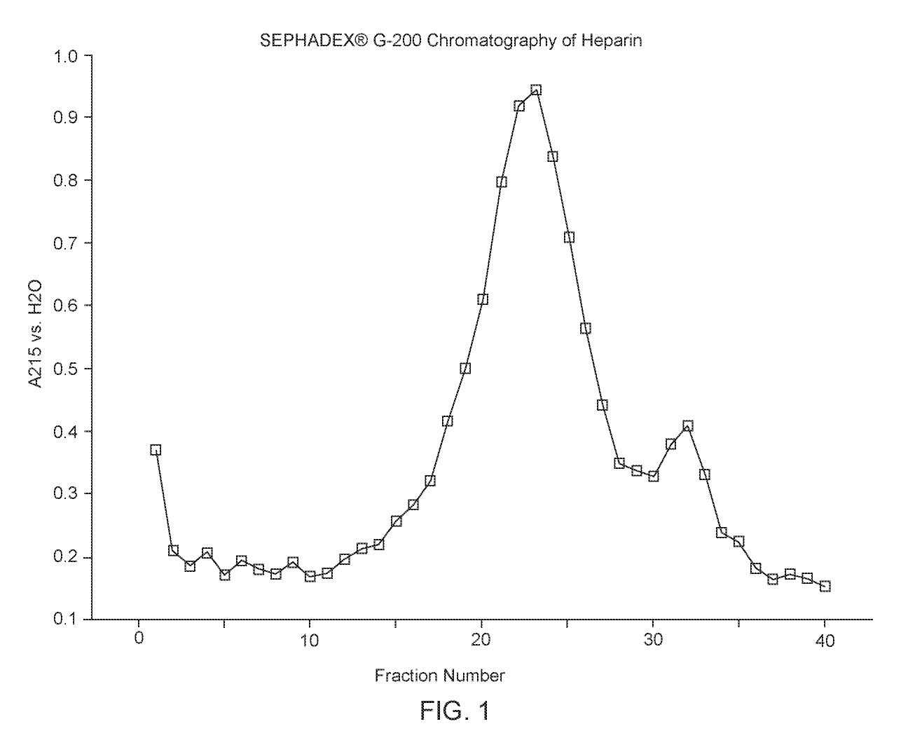

Heparin (0.5 ml of 10,000 I.U./ml of Heparin Leo®) was filtered in a 49 cm by 1 cm Sephadex® G-200 chromatography column. The heparin was eluted with 1 M NaCl and 20-drop fractions (1.27 g per fraction) were collected. The absorbencies of each fraction are shown in Table 1 and FIG. 1.

TABLE 1

| | A₂₁₅ |
|---|---|
| Frac. # | (vs. H₂O) |
| 1 | 0.370 |
| 2 | 0.210 |
| 3 | 0.185 |
| 4 | 0.207 |
| 5 | 0.171 |
| 6 | 0.193 |
| 7 | 0.181 |
| 8 | 0.173 |
| 9 | 0.192 |
| 10 | 0.169 |
| 11 | 0.175 |
| 12 | 0.197 |
| 13 | 0.214 |
| 14 | 0.221 |
| 15 | 0.258 |
| 16 | 0.285 |
| 17 | 0.323 |
| 18 | 0.419 |
| 19 | 0.504 |
| 20 | 0.613 |
| 21 | 0.800 |
| 22 | 0.922 |
| 23 | 0.948 |
| 24 | 0.844 |
| 25 | 0.713 |
| 26 | 0.567 |
| 27 | 0.445 |
| 28 | 0.352 |
| 29 | 0.341 |
| 30 | 0.331 |
| 31 | 0.383 |
| 32 | 0.413 |
| 33 | 0.335 |
| 34 | 0.242 |
| 35 | 0.228 |
| 36 | 0.185 |
| 37 | 0.168 |
| 38 | 0.176 |
| 39 | 0.169 |
| 40 | 0.157 |

Fractions 24-30 were pooled. These fractions excluded heparin chains with very low molecular weights (fractions 31-40). Higher molecular weight heparin chains in fractions 1-23 were excluded for the sake of ease of separating unreacted heparin from ATH in subsequent steps. The heparin chains in fractions 24-30 had molecular weights as high as about 18,000 Daltons. Excluding larger heparin chains ensured that the heparin would not overlap with AT and ATH when purifying the product. However, the higher molecular weight heparin chains can be included in the product in other examples.

Another gel filtration of heparin (0.5 ml of 10,000 I.U./ml of Heparin Leo®) on the 49 cm by 1 cm Sephadex® G-200 chromatography column was carried out. Again, the heparin was eluted with 1 M NaCl and 20-drop fractions (1.23 g per fraction) were collected. The absorbencies of each fraction are shown below in Table 2.

TABLE 2

| | A₂₁₅ |
|---|---|
| Frac. # | (vs. H₂O) |
| 1 | 0.155 |
| 2 | 0.157 |
| 3 | 0.160 |
| 4 | 0.157 |
| 5 | 0.155 |
| 6 | 0.169 |
| 7 | 0.160 |
| 8 | 0.159 |
| 9 | 0.158 |
| 10 | 0.160 |
| 11 | 0.178 |
| 12 | 0.177 |
| 13 | 0.205 |
| 14 | 0.221 |
| 15 | 0.244 |
| 16 | 0.269 |
| 17 | 0.304 |
| 18 | 0.367 |
| 19 | 0.460 |
| 20 | 0.573 |
| 21 | 0.691 |
| 22 | 0.792 |
| 23 | 0.834 |
| 24 | 0.803 |
| 25 | 0.711 |
| 26 | 0.592 |
| 27 | 0.476 |
| 28 | 0.386 |
| 29 | 0.333 |
| 30 | 0.321 |
| 31 | 0.350 |
| 32 | 0.376 |
| 33 | 0.330 |
| 34 | 0.259 |
| 35 | 0.190 |
| 36 | 0.170 |
| 37 | 0.157 |
| 38 | 0.155 |
| 39 | 0.205 |
| 40 | 0.169 |

Results of the chromatography in Table 2 were similar in elution profile to those from the first gel filtration of heparin in Table 1 above. Fractions 24-30 were pooled and combined with the pooled fractions from the first chromatography whose results are given in Table 1. The combined pooled fractions were dialyzed vs. H₂O at 4° C. and then freeze-dried.

### Example 2

### Reaction of Heparin with AT

Human AT was pressure-dialyzed to a concentration of 13.87 milligrams/ml and then further dialyzed against 0.02 M phosphate 0.15 M NaCl pH 7.3 at 4° C., followed by storage at −60° C. after dialysis. 19.12 mg of the heparin fractions freeze-dried in Example 1 above was dissolved in 1 ml of 0.3 M disodium phosphate 1 M NaCl pH 9.5 which had been filtered through a sterile 0.2 micron pore size acrodisc. The resultant solution was placed in a 12 mm by 75 mm plastic test tube and 72 microliters of the human AT were added with mixing. The tube was closed with a plastic cap and sealed around the outside of the cap with parafilm. The tube and contents were heated in a water bath at 37° C. for 14 days.

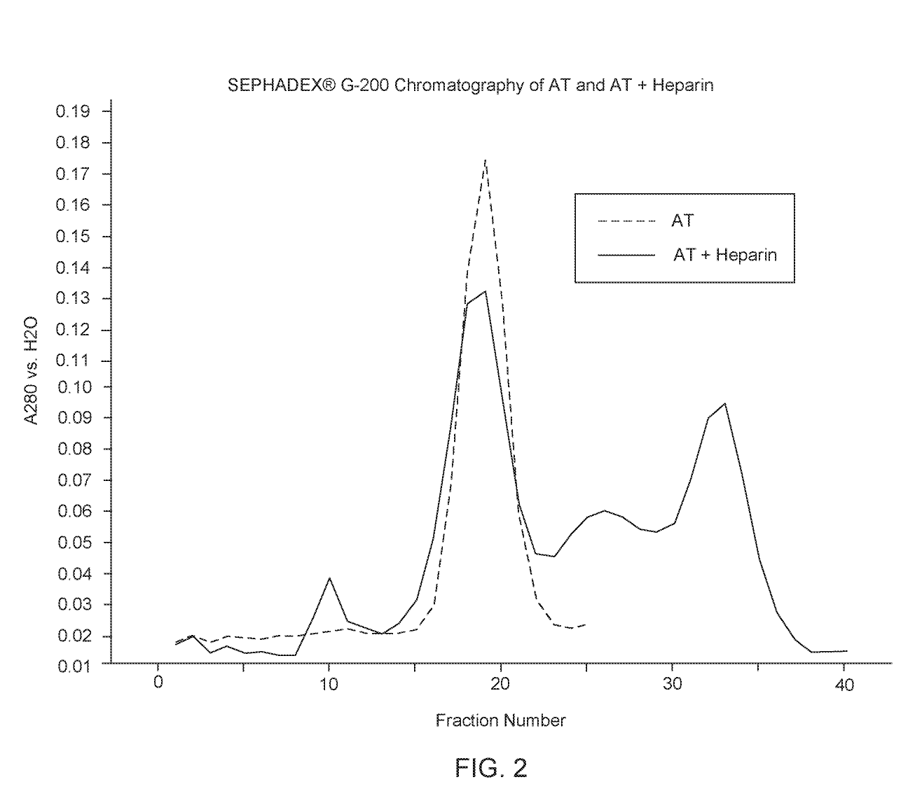

After incubation for 14 days, the mixture of heparin and AT was gel filtered on a 48.5 cm by 1 cm column of Sephadex® G-200 with 1 M NaCl and 20-drop fractions were collected. The absorbences of each fraction are shown in Table 3 and FIG. 2. A separate chromatography of AT alone run on the same Sephadex® G-200 column is co-plotted in FIG. 2 for comparison.

TABLE 3

| | A₂₁₅ | A₂₈₀ |
|---|---|---|
| Frac. # | (vs. H₂O) | (vs. H₂O) |
| 1 | 0.145 | 0.016 |
| 2 | 0.146 | 0.019 |
| 3 | 0.140 | 0.014 |
| 4 | 0.143 | 0.016 |
| 5 | 0.141 | 0.014 |
| 6 | 0.445 | 0.014 |
| 7 | 0.173 | 0.013 |
| 8 | 0.144 | 0.013 |
| 9 | 0.197 | 0.025 |
| 10 | 0.293 | 0.038 |
| 11 | 0.231 | 0.024 |
| 12 | 0.209 | 0.022 |
| 13 | 0.211 | 0.020 |
| 14 | 0.259 | 0.023 |
| 15 | 0.396 | 0.031 |
| 16 | 0.760 | 0.051 |
| 17 | 1.72 | 0.088 |
| 18 | 2.19 | 0.127 |
| 19 | 2.39 | 0.131 |
| 20 | 1.92 | 0.098 |
| 21 | 1.006 | 0.062 |
| 22 | 0.645 | 0.046 |
| 23 | 0.549 | 0.045 |
| 24 | 0.583 | 0.052 |
| 25 | 0.592 | 0.058 |
| 26 | 0.559 | 0.060 |
| 27 | 0.486 | 0.058 |
| 28 | 0.403 | 0.054 |
| 29 | 0.347 | 0.053 |
| 30 | 0.329 | 0.056 |
| 31 | 0.373 | 0.070 |
| 32 | 0.456 | 0.090 |
| 33 | 0.427 | 0.095 |
| 34 | 0.314 | 0.072 |
| 35 | 0.236 | 0.044 |
| 36 | 0.189 | 0.027 |
| 37 | 0.147 | 0.018 |
| 38 | 0.136 | 0.014 |
| 39 | 0.130 | 0.014 |
| 40 | 0.133 | 0.014 |

Fractions 14-16 containing the ATH product were pooled and pressure-dialyzed vs. 0.15 M NaCl to a final mass of 0.74832 g.

### Example 3

### Inhibition of Thrombin Activity by ATH

Experiments to assess the reaction of thrombin with the pressure-dialyzed, pooled fractions of ATH from Example 2 were performed. In each reaction, 114 microliters of the material being analyzed were mixed with 5.83 microliters of 20 U bovine IIₐ (thrombin)/ml 0.15 M NaCl in a plastic Eppendorf tube and left at 23° C. for 10 min. After the 10 minute period, 100 microliters of the solution were mixed with a solution of 875 microliters of 0.036 M sodium acetate 0.036 M sodium barbital 0.145 M NaCl pH 7.4 and 25 microliters of 3.125 mg S-2238/ml H₂O in a cuvette at 23° C. as a clock was started. S-2238 is the chromogenic substrate of the thrombin. The absorbance vs. H₂O at 405 nm of the resultant solution was measured every 10 seconds for 5 minutes. The following reactions were carried out:

Reaction 1 (control): 114 microliters of 0.15 M NaCl was analyzed.

Reaction 2: 114 microliters of ATH was analyzed.

Reaction 3: 28.5 microliters of ATH added to 85.5 microliters of 0.15 M NaCl was analyzed.

Reaction 4: 11.4 microliters of ATH added to 102.6 microliters of 0.15 M NaCl was analyzed.

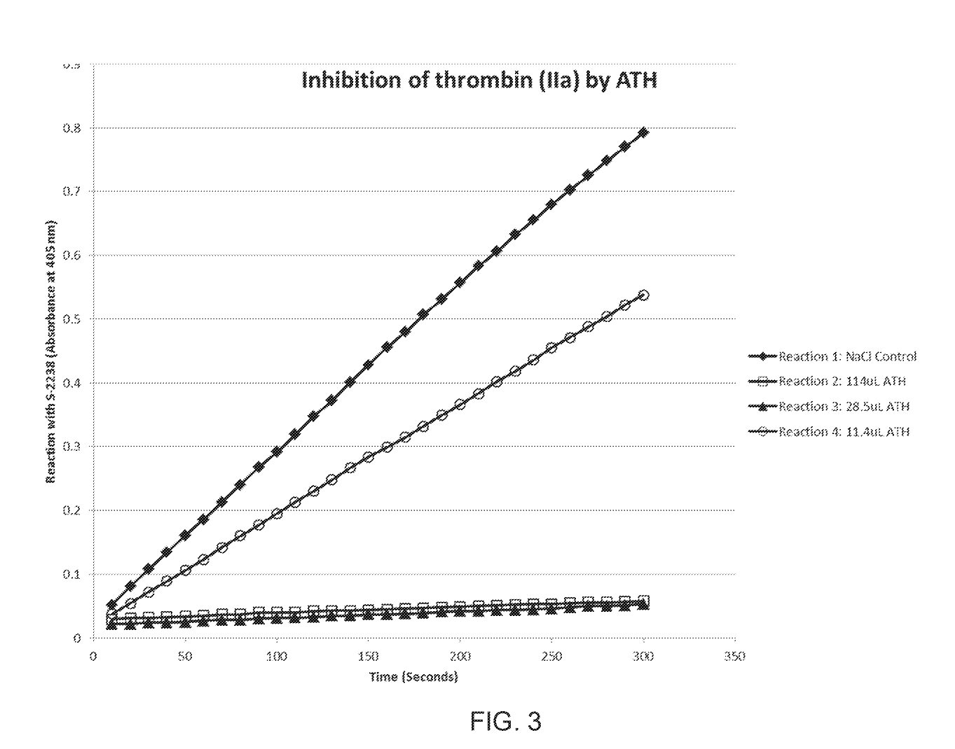

The absorbances at 405 nm are a direct measure of a product cleaved from S-2238 substrate by any thrombin remaining in the cuvette. The absorbances recorded every 10 seconds for each reaction are shown in Table 4 and FIG. 3.

TABLE 4

| Time (seconds) | Reaction 1 | Reaction 2 | Reaction 3 | Reaction 4 |
|---|---|---|---|---|
| 10 | 0.0520 | 0.0290 | 0.0220 | 0.0380 |
| 20 | 0.0810 | 0.0310 | 0.0220 | 0.0540 |
| 30 | 0.1080 | 0.0320 | 0.0240 | 0.0720 |
| 40 | 0.0340 | 0.0330 | 0.0240 | 0.0890 |
| 50 | 0.1610 | 0.0340 | 0.0250 | 0.1060 |
| 60 | 0.1860 | 0.0350 | 0.0270 | 0.1230 |
| 70 | 0.2130 | 0.0370 | 0.0280 | 0.1420 |
| 80 | 0.2400 | 0.0370 | 0.0280 | 0.1600 |
| 90 | 0.2680 | 0.0400 | 0.0300 | 0.1770 |
| 100 | 0.2920 | 0.0400 | 0.0310 | 0.1950 |
| 110 | 0.3190 | 0.0400 | 0.0320 | 0.2130 |
| 120 | 0.3470 | 0.0420 | 0.0330 | 0.2300 |
| 130 | 0.3720 | 0.0430 | 0.0350 | 0.2480 |
| 140 | 0.4010 | 0.0430 | 0.0350 | 0.2670 |
| 150 | 0.4280 | 0.0440 | 0.0370 | 0.2840 |
| 160 | 0.4560 | 0.0450 | 0.0370 | 0.2990 |
| 170 | 0.4800 | 0.0460 | 0.0380 | 0.3150 |
| 180 | 0.5070 | 0.0470 | 0.0390 | 0.3320 |
| 190 | 0.5310 | 0.0480 | 0.0410 | 0.3490 |
| 200 | 0.5570 | 0.0490 | 0.0420 | 0.3660 |
| 210 | 0.5830 | 0.0500 | 0.0420 | 0.3830 |
| 220 | 0.6060 | 0.0510 | 0.0440 | 0.4020 |
| 230 | 0.6320 | 0.0520 | 0.0440 | 0.4180 |
| 240 | 0.6550 | 0.0530 | 0.0450 | 0.4360 |
| 250 | 0.6800 | 0.0530 | 0.0460 | 0.4550 |
| 260 | 0.7020 | 0.0550 | 0.0480 | 0.4710 |
| 270 | 0.7250 | 0.0560 | 0.0500 | 0.4880 |
| 280 | 0.7480 | 0.0560 | 0.0500 | 0.5040 |
| 290 | 0.7700 | 0.0570 | 0.0510 | 0.5220 |
| 300 | 0.7920 | 0.0580 | 0.0530 | 0.5380 |

The data from these four reactions show that even small volumes of the ATH concentrate are able to neutralize the thrombin activity compared to the control reaction (Reaction 1) with only 0.15 M NaCl.

### Example 4

### Clot Time with ATH

10 microliters of 20 U bovine IIₐ (thrombin)/ml 0.15 M NaCl was mixed with either: 90 microliters of 0.15 M NaCl, 85 microliters of 0.15 M NaCl plus 5 microliters of the ATH concentrate from Example 2 above, or 80 microliters of 0.15 M NaCl plus 10 microliters of ATH concentrate. The mixtures were heated at 37° C. for 1 minute in a plastic tube. Then 100 microliters of 0.2 g human fibrinogen/100 ml 0.15 M NaCl was mixed in as a clock was started. The time was recorded at the first appearance of a clot on the end of a wire loop used for agitation. In successive trials, the 90 microliters of pure 0.15 M NaCl gave clot times of 26.2 seconds, 25.2 seconds, and 26.0 seconds. The 85 microliters of 0.15 M NaCl plus 5 microliters of ATH concentrate gave clot times of 34.0 and 33.8 seconds. The 80 microliters of 0.15 M NaCl plus 10 microliters of ATH concentrate gave clot times of 39.2 and 39.6 seconds. The longer clot times indicate reduced thrombin activity in the reactions with the ATH concentrate.

### Example 5

### Clot Time with ATH

100 microliters of 0.2 g human fibrinogen/100 ml 0.15 M NaCl was mixed with either: 90 microliters of 0.15 M NaCl, 85 microliters of 0.15 M NaCl plus 5 microliters of the ATH concentrate from Example 2 above, or 80 microliters of 0.15 M NaCl plus 10 microliters of ATH concentrate. The mixtures were heated at 37° C. for 1 minute in a plastic tube. Then 10 microliters of 20 U bovine IIₐ (thrombin)/ml 0.15 M NaCl was mixed in as a clock was started. The time was recorded at the first appearance of a clot on the end of a wire loop used for agitation. In successive trials, the 90 microliters of pure 0.15 M NaCl gave clot times of 25.8 and 26.0 seconds. The 85 microliters of 0.15 M NaCl plus 5 microliters of ATH concentrate gave clot times of 30.6 and 31.2 seconds. The 80 microliters of 0.15 M NaCl plus 10 microliters of ATH concentrate gave clot times of 37.2 and 35.2 seconds. The longer clot times indicate reduced thrombin activity in the reactions with the ATH concentrate.

### Example 6

### Lyophilizing in High Salt Solution

ATH was prepared as described in Examples 1 and 2 above. Fractions 13-16 (20 drops per fraction, each weighing about 1.2 g to 1.3 g) containing ATH that was eluted from the Sephadex® G-200 with 1 M NaCl were pooled and lyophilized. The lyophilized material was then resuspended in 0.5 ml of water and dialyzed against a 0.15 M NaCl solution. The resuspended ATH was then tested for inhibition of thrombin activity. 3 reactions were performed using: a buffer (0.036 M sodium acetate 0.036 M sodium barbital 0.145 M NaCl pH 7.4), resuspended ATH, a solution of AT at 13.87 micrograms/ml of 0.15 M NaCl, a solution of heparin (similar to that used to make ATH) at 10 micrograms/ml of 0.15 M NaCl, a solution of S-2238 at 3.125 mg/ml H₂O, and a solution of bovine IIₐ (thrombin) at 10 U IIₐ/ml 0.15 M NaCl.

Reaction 1: 114 microliters buffer, 5.83 microliters IIₐ.

Reaction 2: 104.5 microliters buffer, 9.6 microliters resuspended ATH, 5.83 microliters IIₐ.

Reaction 3: 55.0 microliters buffer, 32.9 microliters AT solution, 26.3 microliters heparin solution, 5.83 microliters IIₐ.

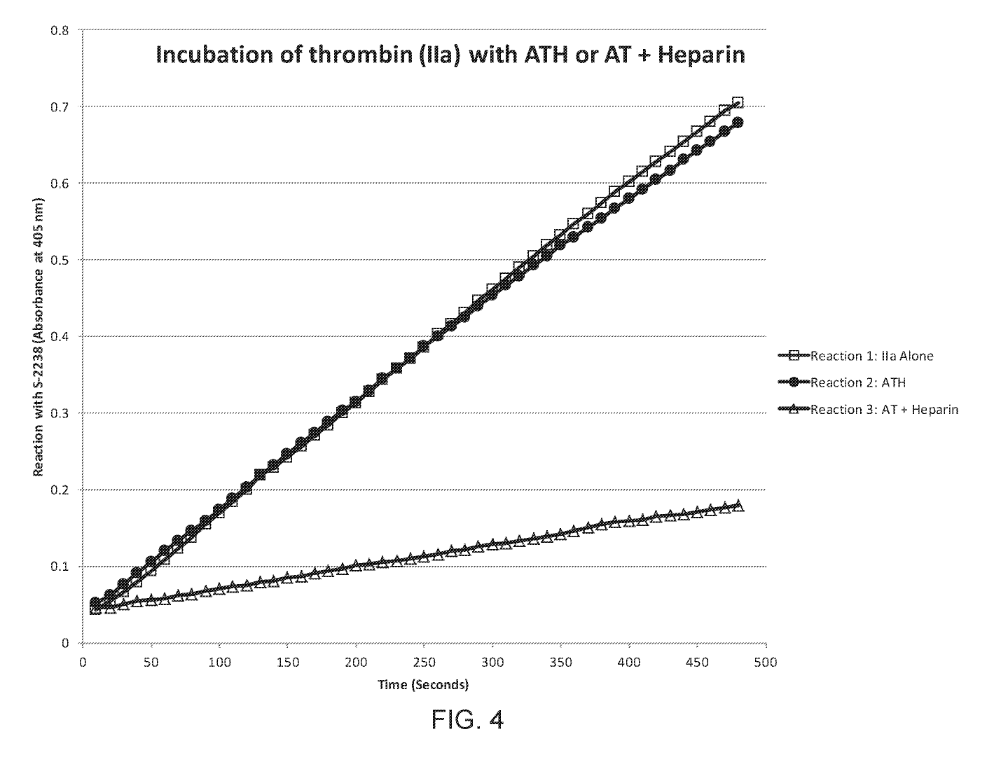

The ingredients were added, in the order shown for each reaction above, into a plastic tube with mixing after each addition. After 10 minutes incubation at 23° C., a 100 microliter aliquot of the reaction was taken and mixed into a solution containing 25 microliters S-2238 plus 875 microliters buffer in a cuvette as a clock was started. Absorbance readings vs. H₂O were taken at 405 nm every 10 seconds. The results of the reactions are shown in FIG. 4. The results show that, in contrast to the AT plus heparin mixture, the resuspended ATH was incapable of inhibiting thrombin. There was no significant reduction in thrombin activity when the resuspended ATH was mixed with the thrombin compared to the thrombin alone (shown as open squares in FIG. 4).

The same resuspended ATH was also tested by combining the resuspended ATH with thrombin and human plasma. A volume of buffer, ATH and/or a sample of heparin fraction (similar to that used to make ATH), and a volume of bovine IIₐ (thrombin) were mixed in a 6 mm by 50 mm borosilicate glass tube at 37° C. After 1 minute, a volume of human plasma (brought to 23° C. just before use) was added with mixing as a clock was started. The time was recorded for first appearance of a clot on the end of a wire loop used for agitation. For each reaction, the volume of bovine IIₐ was 10 microliters of 15 U IIₐ/ml 0.15 M NaCl, and the volume of human plasma was 100 microliters. The volumes of the other components and the clot times are shown in Table 5.

TABLE 5

| | | | 1/10 dilution | heparin | heparin | |
|---|---|---|---|---|---|---|
| | | | in 0.15M | fraction at | fraction at | |
| | | | NaCl of | 10 micrograms/ml | 0.1 micrograms/ml | |
| | buffer | ATH | ATH | 0.15M NaCl | 0.15M NaCl | clot time |
| Reaction # | (microliters) | (microliters) | (microliters) | (microliters) | (microliters) | (seconds) |
| 1 | 90 | 0 | 0 | 0 | 0 | 22.0, 21.8, |
| | | | | | | 22.6 |
| 2 | 63.7 | 0 | 0 | 26.3 | 0 | >120 |
| 3 | 82 | 8 | 0 | 0 | 0 | >120 |
| 4 | 87.4 | 0 | 0 | 2.6 | 0 | >120 |
| 5 | 63.7 | 0 | 0 | 0 | 26.3 | 23.0 |
| 6 | 89 | 0 | 0 | 1 | 0 | 29.2, 29.4 |
| 7 | 89 | 1 | 0 | 0 | 0 | >120 |
| 8 | 86.2 | 0 | 3.8 | 0 | 0 | 28.8, 28.6 |

These data confirm that, although the resuspended ATH did not have activity on its own to inhibit thrombin, the heparin chains of the resuspended ATH were able to catalyze inhibition of thrombin through the exogenous AT found in the human plasma. Clot times for the reactions including the ATH were vastly increased to over 120 seconds. Therefore, there was clearly a sufficient amount of ATH present to inhibit thrombin, but the ATH did not have any activity to inhibit thrombin on its own. These data suggest that the activity of the ATH was destroyed by lyophilizing the ATH in a high (concentrated) salt solution.

### Example 7

### Recycling AT

ATH was synthesized by conjugating AT and unfractionated heparin. The yield from this preparation was 35.28%, defined as the percentage of the starting AT which was recovered as ATH. This left 100-35.28=64.72% of the original AT that was uncomplexed. After the conjugation, leftover unconjugated AT was separated. This unconjugated AT was then used in an additional synthesis in which the recycled AT was reacted with additional heparin to form ATH. Of the recycled AT, 58.59% was converted into ATH. Therefore, of the 64.72% AT which was uncomplexed in the first ATH preparation, a further 58.59% was converted into ATH in the second ATH synthesis. Thus, a further 64.72×58.59/100=37.92% of the original starting AT used in the first ATH preparation was yielded as ATH by recycling the unbound AT in a second ATH synthesis. Finally, combining results from the 2 ATH preparations, the total yield of ATH in terms of the original AT at the start of the first synthesis was 35.28+37.92=73.20%. This total yield of ATH is vastly greater than the maximal 60% yield that is ever obtained in a single ATH synthesis. Moreover, it can easily be seen that unconjugated AT recovered from ATH synthesis with recycled AT could again be used for a third ATH synthesis to boost the conjugate yield in terms of original AT even further.

It is to be understood that the above-referenced arrangements are illustrative of the application for the principles of the present disclosure. Thus, while the present technology has been described above in connection with the exemplary embodiments, it will be apparent to those of ordinary skill in the art that numerous modifications and alternative arrangements can be made without departing from the principles and concepts of the disclosure as set forth in the claims.

What is claimed is:

1. A composition for preventing thrombogenesis, comprising: antithrombin and heparin, wherein at least 50 wt % of the heparin is conjugated to antithrombin, and wherein at least 98 wt % of the heparin in the composition has a molecular weight greater than 3,000 Daltons.

2. The composition of claim 1, wherein at least 95 wt % of the heparin in the composition has a molecular weight greater than 5,000 Daltons.

3. The composition of claim 1, wherein at least 95 wt % of the heparin includes chains containing 18 monosaccharides or more.

4. The composition of claim 1, wherein the heparin includes chains containing a pentasaccharide sequence, as follows:

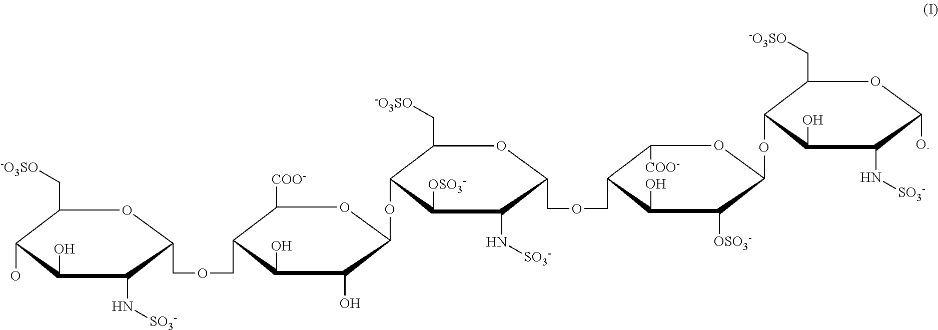

5. The composition of claim 4, wherein, in addition to the pentasaccharide sequence, at least 95 wt % of the heparin includes chains containing 18 monosaccharides or more.

6. The composition of claim 1, wherein at least 95 wt % of the heparin in the composition has a molecular weight less than 30,000 Daltons.

7. The composition of claim 1, wherein at least 95 wt % of the heparin in the composition has a molecular weight less than 20,000 Daltons.

8. The composition of claim 1, wherein the heparin is conjugated to the antithrombin through a linking agent.

9. The composition of claim 1, wherein at least 99 wt % of the heparin in the composition has a molecular weight greater than 3,000 Daltons and less than 30,000 Daltons.

10. The composition of claim 1, wherein at least 75 wt % of the heparin is conjugated to antithrombin.

11. The composition of claim 1, wherein at least 90 wt % of the heparin is conjugated to antithrombin.

12. A method of making a composition for preventing thrombogenesis, comprising:
conjugating antithrombin with heparin outside a body of a subject to form an antithrombin-heparin conjugate;
formulating the antithrombin-heparin conjugate in a solution, comprising:
i) only water and the antithrombin-heparin conjugate,
ii) water, from 0.01-0.09 molar alanine, and the antithrombin-heparin conjugate, or
iii) water, mannitol, and antithrombin-heparin conjugate; and
lyophilizing the solution to form a lyophilized antithrombin-heparin conjugate.

13. The method of claim 12, wherein at least 98 wt % of the heparin in the composition has a molecular weight greater than 3,000 Daltons.

14. The method of claim 12, wherein at least 95 wt % of the heparin in the composition has a molecular weight greater than 5,000 Daltons.

15. The method of claim 12, wherein at least a portion of the heparin with a molecular weight less than 3,000 Daltons are removed prior to the conjugating step.

16. The method of claim 15, wherein the portion of the heparin is removed by a process selected from dialysis, diafiltration, gel filtration, electrophoresis, and combinations thereof.

17. The method of claim 12, wherein at least 99 wt % of the heparin with a molecular weight less than 3,000 Daltons is removed prior to the conjugating step.

18. The method of claim 17, wherein the at least 99 wt % of the heparin is removed by a process selected from dialysis, diafiltration, gel filtration, electrophoresis, and combinations thereof.

19. The method of claim 12, wherein the antithrombin-heparin conjugate retains at least 80% of its thrombin inhibiting activity after lyophilization and resuspension.

20. A composition for preventing thrombogenesis, comprising: an aqueous solution of antithrombin-heparin conjugate, wherein the antithrombin-heparin conjugate is present at a concentration of 9-11 mg/mL with respect to the entire volume of the solution.

21. A method of making a composition for preventing thrombogenesis, comprising conjugating antithrombin with heparin to form an antithrombin-heparin conjugate composition with at least a 60 wt % yield of antithrombin-heparin conjugate based on a starting concentration of antithrombin used to make the antithrombin-heparin conjugate.

22. The method of claim 21, wherein the at least 60 wt % yield is at least partially achieved by recycling unconjugated antithrombin and reacting the unconjugated antithrombin with additional heparin.

23. The method of claim 21, wherein the at least 60 wt % yield is at least partially achieved by introducing an Amadori rearrangement catalyst such that the catalyst is present with the antithrombin and heparin during conjugation.

24. The method of claim 23, wherein the catalyst is selected from the group consisting of 2-hydroxypyridine, tertiary amine salts, ethyl malonate, phenylacetone, acetic acid, and combinations thereof.

25. The method of claim 21, wherein the at least 60 wt % yield is at least partially achieved by conjugating the antithrombin with the heparin in an Amadori rearrangement accelerating solvent system.

26. The method of claim 25, wherein the solvent system comprises a solvent selected from water, formamide, dimethylformamide, dioxane, ethanol, dimethylsulfoxide, pyridine, acetic acid, trimethylamine, trimethylamine, and combinations thereof.

27. The method of claim 21, wherein the at least 60 wt % yield is at least partially achieved by conjugating the antithrombin with the heparin through a linking agent.

28. The method of claim 27, wherein the linking agent is a molecule containing a hydrazine group and an amino group.

29. The method claim 21, wherein the antithrombin heparin conjugate is concentrated by pressure dialysis.

30. The method of claim 21, further comprising steps of:
formulating the antithrombin-heparin conjugate in a solution, comprising:
i) only water and the antithrombin-heparin conjugate,
ii) water, from 0.01-0.09 molar alanine, and the antithrombin-heparin conjugate, or
iii) water, mannitol, and the antithrombin-heparin conjugate; and
lyophilizing the solution to form a lyophilized antithrombin-heparin conjugate.

31. The method of claim 21, wherein the yield is at least 80 wt %.

32. A composition for preventing thrombogenesis, comprising antithrombin, heparin, and antithrombin-heparin conjugate, wherein the antithrombin-heparin conjugate composition has at least a 60 wt % yield of antithrombin-heparin conjugate based on a starting concentration of antithrombin used to make the antithrombin-heparin conjugate.

33. The composition of claim 32, wherein prevention of thrombogenesis utilizes a dosage of the antithrombin-heparin conjugate that is less than 25% by weight of a dosage of heparin used alone to prevent thrombogenesis with the same effectiveness.

34. The composition of claim 32, wherein at least 50 wt % of the heparin is conjugated to antithrombin, and wherein at least 98 wt % of the heparin in the composition has a molecular weight greater than 3,000 Daltons.

35. The composition of claim 32, wherein at least 95 wt % of the heparin in the composition has a molecular weight greater than 5,000 Daltons.

36. The composition of claim 32, wherein at least 95 wt % of the heparin includes chains containing 18 monosaccharides or more, or the heparin includes chains containing a pentasaccharide sequence, as follows: 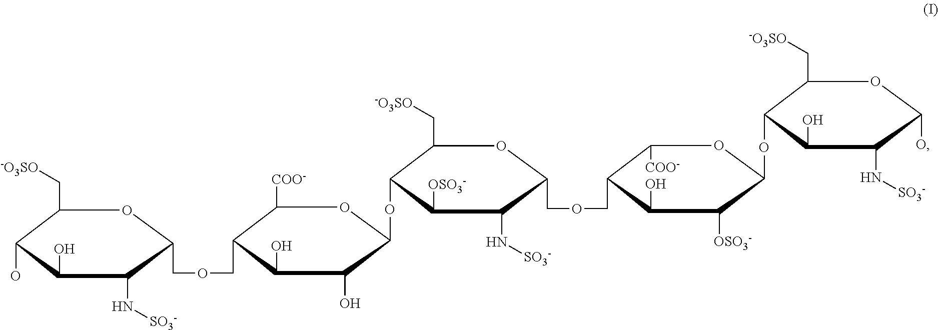
or both.

37. The composition of claim 32, wherein at least 95 wt % of the heparin in the composition has a molecular weight less than 30,000 Daltons.

38. The composition of claim 32, wherein at least 95 wt % of the heparin in the composition has a molecular weight less than 20,000 Daltons.

39. The composition of claim 32, wherein at least 99 wt % of the heparin in the composition has a molecular weight greater than 3,000 Daltons and less than 30,000 Daltons.

40. The composition of claim 32, wherein at least 75 wt % of the heparin is conjugated to antithrombin.

41. The composition of claim 32, wherein at least 90 wt % of the heparin is conjugated to antithrombin.

42. A method of treating a condition or disease, comprising:
administering an antithrombin-heparin conjugate composition to a mammal in need thereof, said antithrombin-heparin conjugate composition, comprising:
i) at least 50 wt % of the heparin conjugated to antithrombin, and wherein at least 98 wt % of the heparin in the composition has a molecular weight greater than 3,000 Daltons; or
ii) an aqueous solution of antithrombin-heparin conjugate, wherein the antithrombin-heparin conjugate is present at a concentration of 9-11 mg/mL with respect to the entire volume of the solution; or
iii) antithrombin, heparin, and antithrombin-heparin conjugate, wherein the antithrombin-heparin conjugate composition has at least a 60 wt % yield of antithrombin-heparin conjugate based on a starting concentration of antithrombin used to make the antithrombin-heparin conjugate.

43. The method of claim 42, wherein administering the antithrombin-heparin conjugate composition comprises administering a dose of antithrombin-heparin conjugate that is less than 25% by weight of a dose of heparin used to treat the condition or disease with the same effectiveness.

44. The method of claim 42, wherein administering the antithrombin-heparin conjugate composition comprises administering a dose of antithrombin-heparin conjugate that is from 0.001 to 50 mg per kilogram body weight of the mammal per day.
